SEQUENCE LISTING (4 sequences)

SEQ ID NO: 1 - protein, 11 aa, Homo sapiens
TINPAVDHCCK

SEQ ID NO: 2 - protein, 8 aa, Homo sapiens
TEDTIFLR

SEQ ID NO: 3 - protein, 11 aa, Homo sapiens
ENFLFLTPDCK

SEQ ID NO: 4 - protein, 10 aa, Homo sapiens
GQYCYELDEK

(19) United States
(12) Patent Application Publication
(10) Pub. No.: US 2012/0107858 A1
(43) Pub. Date: May 3, 2012

(54) CANCER DIAGNOSIS METHOD USING THE GLYCOSYLATION OF A GLYCOPROTEIN

(75) Inventors: Jong Shin Yoo, Seoul (KR); Yeong Hee Ahn, Chungcheongbuk-do (KR); Ju Yeon Lee, Daejeon-shi (KR); Jin Young Kim, Gyeonggi-do (KR)

(73) Assignee: KOREA BASIC SCIENCE INSTITUTE, Daejeon-shi (KR)

(21) Appl. No.: 13/266,893

(22) PCT Filed: Nov. 19, 2009
(86) PCT No.: PCT/KR2009/006836

§ 371 Date: Oct. 28, 2011

(30) Foreign Application Priority Data
May 7, 2009 (KR) 10-2009-0039602

(51) Int. Cl. C12Q 1/37; C07K 16/18

(52) U.S. Cl. 435/23; 530/387.9

(57) ABSTRACT

The present invention relates to a cancer diagnosis method using peptides containing information on the glycosylation of a glycoprotein involving cancer development. More particularly, the present invention relates to a cancer diagnosis method which obtains peptides from the glycoprotein involving cancer development through a hydrolysis process using an enzyme, and quantitatively detects, from among the thus-obtained peptides, glycosylation-related specific peptides which are influenced by the glycosylation of proteins and show specific quantitative changes in the hydrolysis process, to thereby select glycosylation-related specific peptides which show specific quantitative changes in accordance with cancer development. The cancer diagnosis method of the present invention uses the thus-selected glycosylation-related specific peptides as a marker.

### BACKGROUND OF THE INVENTION

1. Field of the Invention

The present invention relates to a selection method of peptides containing information on glycosylation of glycoprotein involved in cancer development, and a cancer diagnosis method using the selected peptides.

2. Description of the Related Art

Protein is an important element involved in various life support activities being progressed in an organism. Thus, studies on functions and identification of endogenous proteins are very important to understand such proteins involved in vital activity and further to establish an early diagnosis and treatment method of disease based on the understanding of functions of such proteins.

Proteins play an important role in life support activity and are through post-translational modification by signal transduction whenever necessary. The most representative post-translational modification processes are glycosylation and phosphorylation. In particular, regarding glycosylation of a glycoprotein, many monosaccharides existing on the surface of cell membrane are passed through the cell membrane by signal transduction, leading to glycosylation of a required protein by N-acetylglucosaminyltransferase. Such glycoprotein plays an important role as being located on the outer membrane. Once glycoproteins finish their required role, they proceed to glucolysis by glycosidase. However, many glycoproteins or glycolipids located on the surface of cell membrane often experience abnormal glycosylation by the specific signal such as oncogene, etc. Many diseases have been known to be closely related to such abnormal functions of glycosidase and glycosyltransferase triggered by abnormal signal transduction by oncogene (Kim, Y. J., et al., Glycoconj. J., 1997, 14, 569-576, Hakomori, S., Adv. Cancer Res., 1989, 52, 257-331, Hakomori, S., Cancer Res., 1996, 56, 5309-5318).

Glycosylation of a protein is largely divided into two types: One is N-linked glycosylation characterized by glycosylation through side chains of asparagine in the specific nucleotide sequence combination (NXS/T, X is the amino acid except proline) during protein synthesis and the other is O-linked glycosylation characterized by glycosylation through hydroxyl group forming side chains of such amino acids as serine and threonine, etc. Glycan largely observed in glycoprotein is exemplified by glucose (Glc), galactose (Gal), mannose (Man), fucose (Fuc), N-acetylgalactosamine (GalNAc), N-acetylglucosamine (GlucNAc), and N-acetylneuraminic acid (NeuNAc) (Frank Kjeldsen, et al., Anal. Chem. 2003, 75, 2355-2361). Functions of the glycosylated protein such as folding, recognition, and solubility, etc are regulated by diversity of the sugar (Varki, A. et al., Glycobiology 1993, 3, 97-130, Parodi, A. J. et al., Annu. Rev. Biochem. 2000, 69, 69-93).

Difference of glycosylation in protein samples obtained from normal group and patient group can be an important clue to distinguish patient group from normal group. Up to date, many analysis methods have been reported to tell the difference of glycoprotein. Protein profiling is an example of a method to screen difference in glycosylation of a protein, which analyzes glycans obtained from hydrolysis of glycoproteins by using mass spectrometer (Cooke C. L. et al., Anal. Chem., 2007, 79, 8090-8097). However, this method has a disadvantage of losing information on the glycosylated isoform in relation to characteristics of glycosylation and glycosylated structure in the specific glycosylation site of each protein, because this method can only analyze the averaged glycan states composed of all kind of glycans originated and pooled from different proteins and glycosylation sites. This conventional method enables distinguishment of normal from patient based on the difference in general profiling, but provides only limited information because information on glycoprotein, glycosylation site, and glycosylated isoform, etc is lost.

In another method, intact glycoproteins having high molecular weight can be enriched. For the enrichment, various lectins including ConA (mannose), WGA (N-acetylglucosamine), Jacalin (galactose), SNA (sailic acid), AAL (fucose) or multilectin prepared by mixing the diverse lectins can be used (Yang, Z. et al., J. Chromatogr, A, 2004, 1053, 79-88, Wang, Y. et al., Glycobiology, 2006, 16, 514-523). Or hydrazide can be used, which is called glycan-capturing (Zhang H. et al., Nat. Biotechnol., 2003, 21, 660-666). These methods can be used not only for the enrichment of a glycoprotein but also for the enrichment of a glycopeptide. To increase liability of qualitative analysis, peptides obtained from hydrolysis of diverse glycoproteins in which sugars have been detached by a required process are used for qualitative analysis or isotope labeled reagent is used for quantitative analysis (Tian Y., et al., Nat. Protocols, 2007, 2, 334-339). However, it is not possible with this method to distinguish different glycan-isoforms having different glycosylated structures.

Plasma proteome is composed of at least 50,000 constituents and the abundance of protein components is very dynamic (1˜10¹²). So, a protein biomarker candidate existing at a very low concentration is hard to detect and analyze quantitatively by liquid chromatography-mass spectrometry (LC/MS/MS) (Anderson N. L. et al., Mol. Cell Proteomics. 2002, 1, 845-867). To minimize complexity of the sample for efficient detection of a disease biomarker in serum, protein removal column (ex, MARS, Multiple Affinity Removal System) is first used to eliminate high abundant proteins taking about 90% or more of plasma proteome, for example albumin, IgG, IgA, transferrin, and haptoglobin, and then the resultant proteome is used. Or, proteome which has not been through such elimination process can also be used. But generally those proteins taking 90% of serum are eliminated to prepare target proteome. To obtain glycoprotein, multilectin is used to enrich glycoprotein only without using massive protein removal column, or massive protein removal column and multilectin can be used stepwise. Other massive protein removal columns or lectin having corresponding constitution can also be used. So, the prepared plasma proteome proceeds to purification process by acetone precipitation or MWCO (molecular weight cut-off) method to eliminate salts used in the glycoprotein collection process.

Those proteins or peptides having high molecular weight can be analyzed by mass spectrometer. Mass spectrometer has three different functional parts, which are source, analyzer, and detector. A sample is ionized in the source part. The ionized sample is separated according to the ratio of mass/charge in the analyzer. The separated ions are detected in the detector. There are two main soft ionization methods for ionization of proteins or peptides having high molecular weight. One is ESI (electrospray ionization) that enables the detection of biomolecules having high molecular weight without destroying bindings, compared with the conventional ionization methods. The other method is MALDI (matrix-assisted laser desorption ionization). In the method of ESI, sample complexity can be reduced by combining HPLC or capillary type electrophoresis as the pretreatment process of a sample, in order to reduce unexpected effect of salts or impurities. The analyzer part of mass spectrometer is composed of IT-LIT (iontrap-linear ion trap), Q-Q-TOF (quadruple-quadruple-time of flight), TOF-TOF (time of flight-time of flight), FT-ICR (Fourier Transform Ion Cyclotron Resonance), Q-Q-Q (quadruple-quadruple-quadruple), QQ-LIT (quadruple-quadruple-iontrap-linear ion trap), and LIT-Orbitrap (linear ion trap-orbitrap), etc. In general, single or mixed type which gives advantage in identification of fragmented peptide is preferred.

Endogenous high molecular weight proteins are fragmented to peptides by the sample treatment process stated above, followed by analysis using mass spectrometer. Then, identification is performed using search engines such as SEQUEST (http://www.thermo.com), MASCOT (http://www.matrixscience.com), protein expression system (http://www.waters.com), X! tandem (http://proteome.ca/opensource.html), peptideProphet (http://www.proteomecenter.org/software.php), and OMSSA (http://pubchem.ncbi.nlm.nih.gov/omssa/). The results of mass analysis of sample peptides are screened through all sequences reserved in data base by computer, followed by prediction of mass and morphology of imaginary fragments by using algorithm used in the search engines mentioned above, considering protein digestive rules. The predicted result is then compared with the experiment result. The level of consistency between the two results is presented as probability and the target protein is identified based on the probability. To identify a protein by mass spectrometry, the sequence of the target protein has to be saved already in database. Such database of protein sequence is provide by Swiss-Prot, TrEMBL (Translated European Molecular Biology Laboratory), UniProt (Universal Protein Resource), NCBI (National Center for Biotechnology Information), IPI (The International Proteins Index), etc (Diamond1 D. L., et al., Hepatology 2006, 44, 229-308).

Protein labeling using a stable isotope is one of the methods for quantitative analysis of identified proteins using mass spectrometer. For example, most representative ones are ICAT (Isotope-Coded Affinity Tags), ICPL (Isotope Coded Protein Label), the method in which proteins produced by replacing nitrogen source with 15N-isotope in culture medium are analyzed with mass spectrometer, and SILAC (Stable isotope labeling with Amino acids in Cell culture) in which stabilized isotope amino acid is added to culture medium to introduce it into proteins expressed during cell culture. The method of labeling peptides generated by protein hydrolysis, iTRAQ (Isobaric Tags for Relative and Absolute Quantitation), and the method to replace only glycoproteins or hydrolyzed glycopeptides with isotope can also be used (Tian Y., et al., Nat. Protocols, 2007, 2, 334-339).

In addition, a label-free method can be used, which compares normal group with patient group based on the peptides confirmed to be reliable by repeated experiments measuring accurate molecular weight using mass spectrometer without labeling peptides or proteins and examining retention time on liquid chromatography (Silva J. C., et al., Anal. Chem. 2005, 77, 2187-2200, Finney G. L., et al., Anal. Chem. 2008, 80, 961-971). When proteins that exist in a sample at trace level are targeted to screen a biomarker candidate, MRM (multiple reaction monitoring) showing excellent selectivity and sensitivity is used. MRM is performed by two different ways; one is label-free comparative quantitative analysis and the other is absolute quantitative analysis characterized by injecting a stable isotope labeled peptide standard in advance. To perform MRM more efficiently, selection of peptides detected in the target protein only and confirmation of MRM transition of the corresponding peptides by using a specific program and database such as TIQAM (targeted identification for quantitative analysis by MRM) are important factors to be considered (Anderson L, et al., Mol. Cell Proteomics. 2006, 5, 573-588).

Immunoaffinity-MS using SISCAPA (stable isotope standards with capture by anti-peptide antibodies) is the method to obtain the peptide representing the screened biomarker candidate protein in a large scale, to construct antibody recognizing the screened peptide, to isolate the target peptide from mixed peptides using the antibody, and to analyze the target by minimizing sample complexity by MRM. This method can improve LOD (Limit of Detection) and LOQ (Limit of Qualification) of LC/MS/MS. Therefore, this method can take the place of the conventional methods such as ELISA (enzyme-linked immunosorbent assay) and Western blot showing excellent LOD and LOQ but no peptide selectivity (Anderson N L, et al., J Proteome Res. 2004. 3, 235-244). Antigen peptides isolated and enriched selectively by using an antibody can be analyzed as being conjugated with the antibody by immuno-MALDI MS (iMALDI MS) using MALDI mass spectrometer.

The present inventors have studied to develop a novel method to distinguish difference in glycosylation of protein between normal group and liver cancer patient group. As a result, the present inventors confirmed that when glycoprotein is glycosylated, the efficiency of hydrolysis of specific peptides is affected by steric hindrance effect generated by sugar chains taking a huge space, so the resultant peptide level is changed according to the level of glycosylation and the structure of sugar chain nearby. The present inventors also confirmed that such specific peptides involved in glycosylation generated from hydrolysis of glycoproteins are useful for the diagnosis of cancer by quantitative mass spectrometry, and such peptides thereby can be used as a cancer diagnosis marker, leading to the completion of this invention.

### SUMMARY OF THE INVENTION

It is an object of the present invention to provide a method to select glycosylation-related specific peptides for cancer diagnosis by taking advantage of the specific phenomenon that hydrolysis process of the specific peptide is changed according to the changes of sugar chains of the glycoprotein involved in cancer development, and a cancer diagnosis method using the selected specific peptides.

To achieve the above object, the present invention provides a screening method for cancer diagnosis marker characterized by selecting glycosylation-related peptides by taking advantage of the specific phenomenon that the amount of hydrolyzed peptide is quantitatively, specifically changed according to the changes of sugar chains of glycoproteins when the protein isolated/purified from the cancer patient sample is hydrolyzed into peptides using hydrolase.

The present invention also provides a cancer diagnosis method characterized by determining if a subject has high risk of cancer when the subject has glycosylation-related peptides in which the amounts of hydrolyzed peptides are quantitatively, specifically changed according to the changes of sugar chains of glycoproteins when the protein isolated/purified from the subject sample is hydrolyzed into peptides using hydrolase.

The present invention also provides a diagnostic kit for cancer comprising the antibody specifically binding to one or more glycosylation related peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4.

The present invention also provides a biochip for cancer diagnosis on which the antibody specifically binding to one or more glycosylation related peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 is integrated on the solid substrate.

The present invention also provides a use of the antibody specifically binding to any peptide selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 for the preparation of the diagnostic kit for cancer.

In addition, the present invention provides a use of a biomolecule, which can be obtained from the blood sample of a subject, specifically binding to the combination of one or more peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 for the preparation of the biochip for cancer diagnosis.

### ADVANTAGEOUS EFFECT

As explained hereinbefore, the present invention is advantageous for the fast and early diagnosis of cancer via quantitative analysis with specific peptides obtained from the sample of a subject containing information on abnormal glycosylation of protein concerning glycosylation level and sugar chain structure, and this selected specific peptide can be effectively used for the marker for cancer diagnosis.

### BRIEF DESCRIPTION OF THE DRAWINGS

The application of the preferred embodiments of the present invention is best understood with reference to the accompanying drawings, wherein:

FIG. 1 is a schematic diagram illustrating the process of LC/MS/MS with peptide fragments obtained by trypsin digestion of proteome originated from blood plasma taken from both normal and patient groups.

FIG. 2 is a graph illustrating the statistic result of PCA (principle component analysis) with whole peptides of normal and patient groups quantitatively analyzed by LC/MS/MS.

FIG. 3 is a graph illustrating the statistic result of PCA (principle component analysis) with 4 selected specific peptides greatly involved in specificity to distinguish normal group from patient group.

FIG. 4 is a set of graphs illustrating the result of ROC (receiver operating characteristic curve) with 4 selected specific peptides greatly involved in specificity to distinguish normal group from patient group.

FIG. 5 is a schematic diagram illustrating that those 4 selected specific peptides greatly involved in specificity to distinguish normal group from patient group are the peptides closely related to N-linked glycosylation and the efficiency of hydrolysis of glycoprotein depends on glycosylation.

### DESCRIPTION OF THE PREFERRED EMBODIMENTS

Hereinafter, the present invention is described in detail.

The present invention provides a screening method for cancer diagnosis marker characterized by selecting glycosylation-related peptides by taking advantage of the specific phenomenon that the amount of hydrolyzed peptide is quantitatively, specifically changed according to the changes of sugar chains of glycoproteins when the protein isolated/purified from the cancer patient sample is hydrolyzed into peptides using hydrolase.

In this invention, the changes of sugar chains of glycoproteins indicate different aspects of glycosylation of protein shown in cancer patients or those having history of cancer. Such changes can occur at asparagine, threonine, or serine site, and include any changes in glycosylation level and sugar chain structure of each site.

In this invention, the cancer is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto. Cancer is understood to be developed by abnormal signaling and recognition. Such abnormality is attributed largely to glycoproteins exiting on or secreted from cell surface.

In this invention, glycoprotein or peptide used herein can be prepared and used as samples for cancer diagnosis considering cell lines, regions of cell, tissues of organ, drug administration, diet habit, nutrition status, and progress of disease, etc.

Glycosylation site of glycoprotein takes a quite huge steric space, which can affect hydrolysis efficiency of neighboring specific peptides. Therefore, the amount of specific peptide generated by hydrolysis can be affected by the difference or changes of the glycosylation site. Unlike normal samples, cancer samples show abnormal glycosylation such as unnecessary glycosylation or modified glycosylation caused by abnormal signaling, recognition, or adhesion. Such abnormal glycosylation of protein makes difference in hydrolysis of protein, which can be confirmed by quantitative analysis with specifically selected peptides, resulting in distinguishment blood sample of cancer patient from normal blood sample.

Particularly, the screening method for cancer diagnosis marker is preferably composed of the following steps, but not always limited thereto:

1) isolating total protein from the sample obtained from cancer patient;

2) purifying the isolated total protein by using mass protein removal column;

3) preparing hydrolyzed peptide fragment mixture by treating the purified protein with hydrolase;

4) quantitative-analyzing the hydrolyzed peptide fragment mixture;

5) screening those peptides showing significant change in the amount compared with that of the control; and

6) confirming if the selected peptides showing significant change in the amount are originated from glycoprotein.

In the above method, the cancer of step 1) is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

In the above method, the sample of step 1) is preferably blood sample because blood contains every protein secreted from all different organs. The sample herein can be not only blood but also plasma, serum, saliva, urine, cerebrospinal fluid, follicular fluid, breast milk, lens fluid, and pancreatic juice, which all can be good samples for cancer diagnosis using glycoprotein related peptides.

In the above method, the protein of step 1) is not limited in its size, and can be oligopeptide, polypeptide or protein.

In the above method, the purification of step 2) is performed by using mass protein removal column [ex, MARS (Multiple Affinity Removal System)] to minimize complexity of the sample because the density of protein components of the proteome isolated from the sample is very dynamic, making the detection of biomarker candidate proteins and quantitative analysis are difficult, but not always limited thereto.

The protein obtained from the sample is hard to be analyzed at protein level. To analyze the protein, it needs to be hydrolyzed. To do so, pre-treatment such as denaturation, reduction, cysteine alkylation, dephosphorylation, or deglycosylation can be performed.

Isolation/purification of the protein is preferably performed by 1D-gel protein separation, 2D-PAGE, SEC (Size Exclusion Chromatography, FFE system (Free Flow Electrophoresis System), or FFF (Field Flow Fractionation), but not always Limited thereto.

In the above method, the hydrolase of step 3) is preferably one or more enzymes selected from the group consisting of Arg-C, Asp-N, Glu-c, Lys-C, chymotrypsin and trypsin, and more preferably trypsin, but not always limited thereto.

After the pre-treatment, it is preferred that the high molecular proteins or glycoproteins are hydrolyzed to low molecular peptides by using various hydrolases in order to analyze them with mass spectrometer.

In general, to hydrolyze a protein into peptide fragments, trypsin that can digest amide bond between lysine and arginine is mainly used. But, Lys-C that digests lysine site only, Arg-C that digests arginine site only, and Asp-N that digests asparagine site only, etc can also be used selectively or stepwise, according to the purpose.

As a step of pre-treatment, the hydrolyzed peptide fragments preferably proceed to desalting using zip-tip or trap column equipped on LC to be available for automatic elimination of salts, so as to eliminate all the salts that might cause any problem in mass spectrometry, but not always limited thereto.

In the above method, the quantitative analysis of step 4) is preferably performed by the method selected from the group consisting of protein chip analysis, MALDI-TOF (Matrix Assisted Laser Desorption/Ionization Time of Flight Mass Spectrometry), SELDI-TOF (Surface Enhanced Laser Desorption/Ionization Time of Flight Mass Spectrometry), 2-dimensional electrophoresis, liquid chromatography-Mass Spectrometry (LC-MS), Western blot and ELISA, and more preferably performed by electrospray ionization (ESI) directly connected to nano-HPLC, but not always limited thereto.

To compare normal group with patient group, the results of quantitative analysis are statistically analyzed by hierarchical cluster, PCA (principle component analysis), etc. If necessary, normalization can be performed to minimize differences in each analysis batch.

In the above method, the ‘significant change in the amount’ of step 5) indicates either increase or decrease of the amount.

In the above method, the glycoprotein originated peptides of step 6) preferably have glycosylation site within the range of 8 amino acids from hydrolysis site either at N-terminal or C-terminal of the amino acid sequence, but not always limited thereto.

The glycosylation site of glycoprotein takes a quite huge steric space, which can affect hydrolysis efficiency of neighboring specific peptides. Therefore, the amount of specific peptides generated by hydrolysis can be affected by the difference or changes of the glycosylation site. Unlike normal samples, cancer samples show abnormal glycosylation such as unnecessary glycosylation or modified glycosylation caused by abnormal signaling, recognition, or adhesion. Such abnormal glycosylation of protein makes difference in hydrolysis of protein, which can be confirmed by quantitative analysis with specifically selected peptides, resulting in distinguishment of cancer patient blood sample from normal blood sample.

The present invention also provides a cancer diagnosis method characterized by determining if a subject has high risk of cancer when the subject has glycosylation-related peptides in which the amount of hydrolyzed peptide is quantitatively, specifically changed according to the changes of sugar chains of glycoproteins when the protein isolated/purified from the subject sample is hydrolyzed into peptides using hydrolase.

Particularly, the cancer diagnosis method is preferably composed of the following steps, but not always limited thereto:

1) isolating total protein from the sample obtained from a subject;

2) purifying the isolated total protein by using mass protein removal column;

3) preparing hydrolyzed peptide fragment mixture by treating the purified protein with hydrolase;

4) quantitative-analyzing the hydrolyzed peptide fragment mixture;

5) screening those peptides showing significant change in the amount compared with that of the control;

6) confirming if the selected peptides showing significant change in the amount are originated from glycoprotein; and

7) Judging or diagnosing cancer or high risk of cancer to the subject when the peptides showing significant change in the amount are confirmed to be originated from glycoprotein.

In the above method, the cancer is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

In the above method, the sample of step 1) is preferably blood sample because blood contains every protein secreted from all different organs. The sample herein can be not only blood but also plasma, serum, saliva, urine, cerebrospinal fluid, follicular fluid, breast milk, lens fluid, and pancreatic juice, which all can be good samples for cancer diagnosis using glycoprotein related peptides.

In the above method, the protein of step 1) is not limited in its size, and can be oligopeptide, polypeptide or protein.

In the above method, the purification of step 2) is performed by using mass protein removal column [ex, MARS (Multiple Affinity Removal System)] to minimize complexity of the sample because the density of protein components of the proteome isolated from the sample is very dynamic, making the detection of biomarker candidate protein and quantitative analysis are difficult, but not always limited thereto.

The protein obtained from the sample is hard to be analyzed at protein level. To analyze the protein, it needs to be hydrolyzed. To do so, pre-treatment such as denaturation, reduction, cysteine alkylation, dephosphorylation, or deglycosylation can be performed.

Isolation/purification of the protein is preferably performed by 1D-gel protein separation, 2D-PAGE, SEC (Size Exclusion Chromatography, FFE system (Free Flow Electrophoresis System), or FFF (Field Flow Fractionation), but not always Limited thereto.

In the above method, the hydrolase of step 3) is preferably one or more enzymes selected from the group consisting of Arg-C, Asp-N, Glu-c, Lys-C, chymotrypsin and trypsin, and more preferably trypsin, but not always limited thereto.

After the pre-treatment, it is preferred that the high molecular proteins or glycoproteins are hydrolyzed to low molecular peptides by using various hydrolases in order to analyze them with mass spectrometer.

In general, to hydrolyze a protein into peptide fragments, trypsin that can digest amide bond between lysine and arginine is mainly used. But, Lys-C that digests lysine site only, Arg-C that digests arginine site only, and Asp-N that digests asparagine site only, etc can also be used selectively or stepwise, according to the purpose.

As a step of pre-treatment, the hydrolyzed peptide fragments preferably proceed to desalting using zip-tip or trap column equipped on LC to be available for automatic elimination of salts, so as to eliminate all the salts that might cause any problem in mass spectrometry, but not always limited thereto.

In the above method, the quantitative analysis of step 4) is preferably performed by the method selected from the group consisting of protein chip analysis, MALDI-TOF (Matrix Assisted Laser Desorption/Ionization Time of Flight Mass Spectrometry), SELDI-TOF (Surface Enhanced Laser Desorption/Ionization Time of Flight Mass Spectrometry), 2-dimensional electrophoresis, liquid chromatography-Mass Spectrometry (LC-MS), Western blot and ELISA, and more preferably performed by electrospray ionization (ESI) directly connected to nano-HPLC, but not always limited thereto.

To compare normal group with patient group, the results of quantitative analysis are statistically analyzed by hierarchical cluster, PCA (principle component analysis), etc. If necessary, normalization can be performed to minimize differences in each analysis batch.

In the above method, the ‘significant change in the amount’ of step 5) indicates either increase or decrease of the amount.

In the above method, the glycoprotein originated peptides of step 6) preferably have glycosylation site within the range of 8 amino acids from hydrolysis site either at N-terminal or C-terminal of the amino acid sequence, but not always limited thereto.

The said method is qualitative/quantitative analysis method for normal group and patient group without labeling. In the case of quantitative-analyzing proteome by such label-free method, accuracy of mass spectrometer for measurement of molecular weight and reproducible retention time of the mixed peptides separated through LC column are important. So, it is preferred to load the peptides at a regular interval with knowing their exact molecular weight so as to modify their molecular weight after analysis, but not always limited thereto.

In the above method, when quantitative analysis is performed by the label-free method using mass spectrometer, it is required to correct errors among batches of samples. To do so, the peptides obtained by hydrolyzing a standard protein not detected in the sample are used as internal standard material. This internal standard material is added to each sample each time by the same amount, leading to normalization of batch errors, which is a preferred method to reduce errors among batches of quantitative analysis, but not always limited thereto. At this time, any kind of protein can be used as internal standard material as long as it is not detected in the sample to be analyzed, or peptide of protein being detected by mass spectrometry can be used as internal standard material as long as quantitative difference is not observed in between normal group and patient group.

The above method has advantages as follows: the pre-treatment can be performed by the conventional proteome treatment method, any additional step of collecting sugar or glycoprotein is not necessary, it targets those proteins included in the target sample at the detectable concentration according to the recent proteome analysis techniques, and it enables distinguishment of patient group from normal group without isotope substitution that is very complicated and asks high price for quantitative analysis.

In the above method, patient group is distinguished from normal group by quantitative analysis based on the result of investigation of subject sample without labeling. However, various protein labeling methods or hydrolyzed peptide labeling methods can be selectively used.

In the above method, glycoprotein related peptides are fast screened. In cancer patients, glycosylation is abnormally progressed, unlike in normal people, so that such fast screening helps understanding of different life support activities between normal group and patient group and also helps efficient diagnosis of diverse diseases.

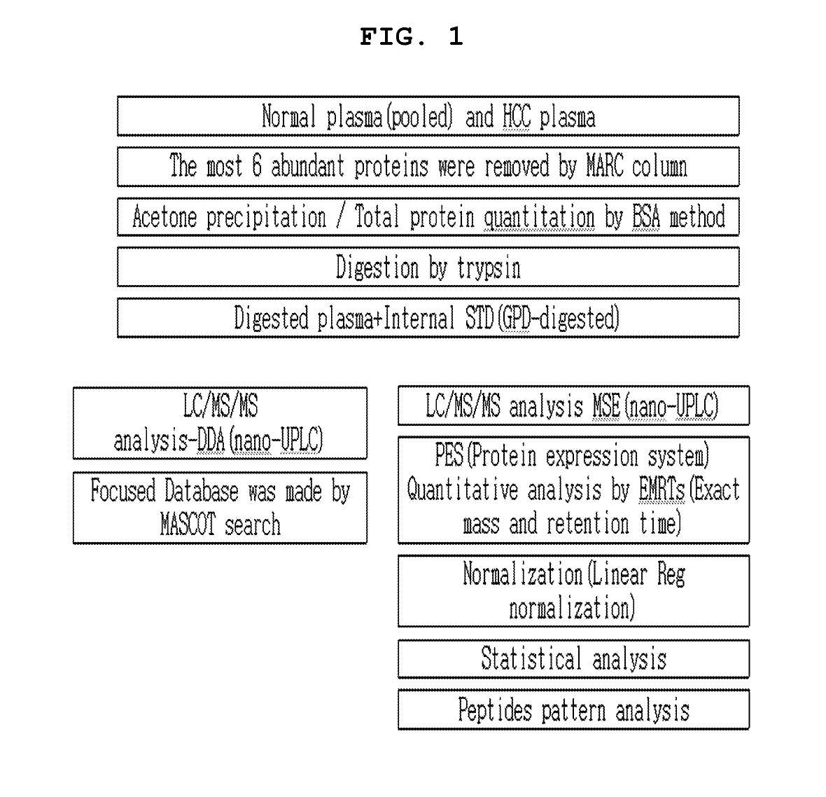

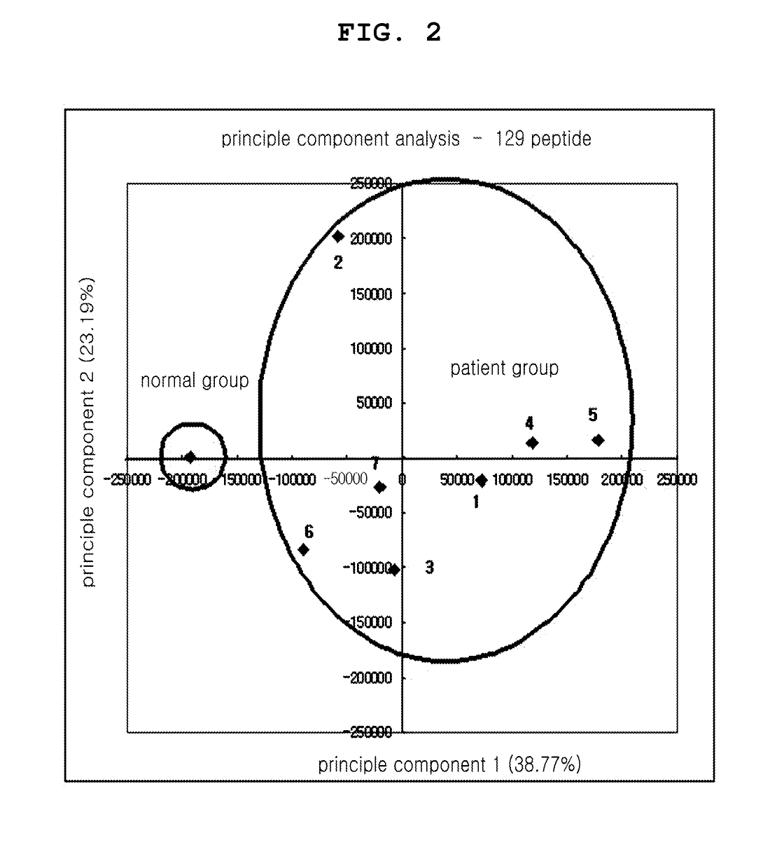

The present inventors isolated proteome from blood of both normal and cancer patient. Mass protein that took at least 90% was eliminated by mass protein removal column, followed by aceton-precipitation to obtain purified proteome. A certain amount of the purified proteome was hydrolyzed by using trypsin to obtain peptide mixture. The peptide mixture obtained from each proteome was analyzed three times by LC/MS/MS. Focus database was constructed with the confirmed 129 peptides, based on which qualitative/quantitative mass spectrometry was performed with each proteome sample (see FIG. 1). PCA (principle component analysis) was performed based on the above results, from which patient group was clearly distinguished from normal group (see FIG. 2).

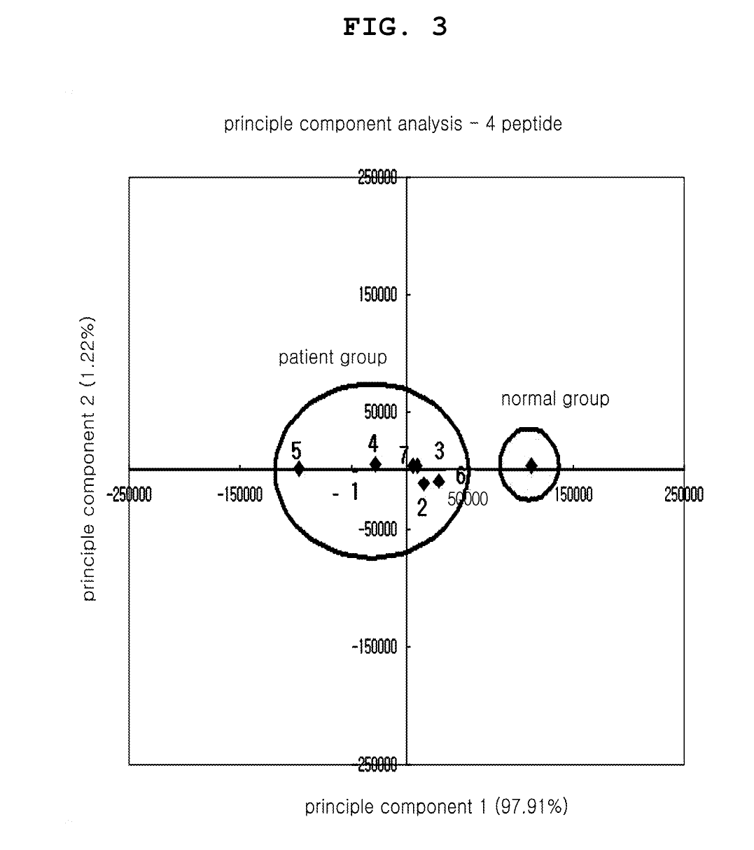

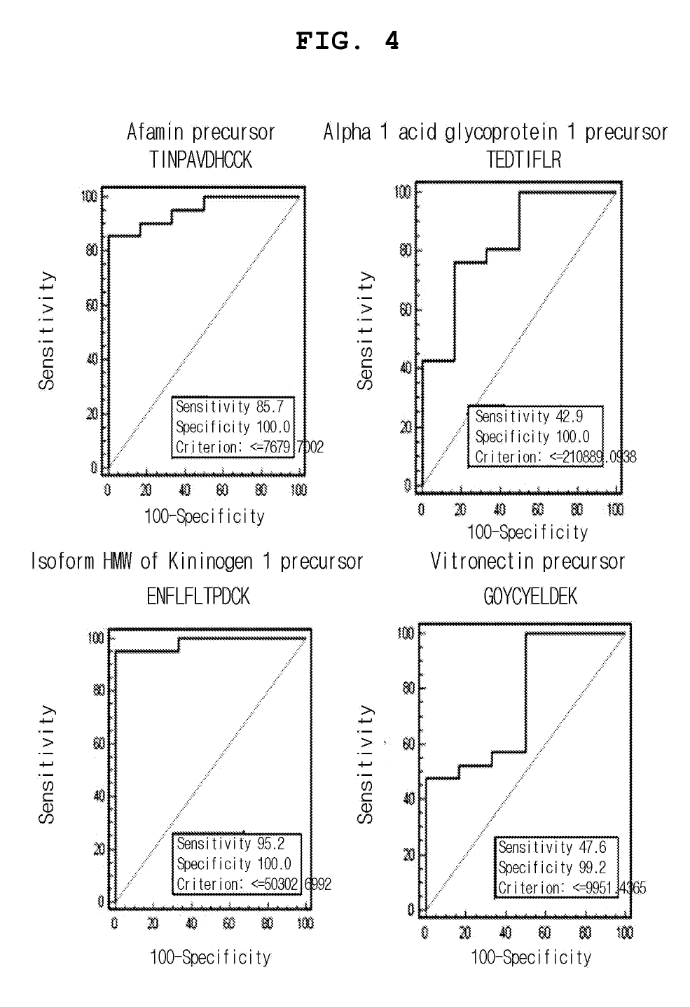

To screen those peptides specifically demonstrating difference between in normal group and in patient group, peptide pattern of each protein was analyzed. As a result, 4 specific peptides originated from glycoprotein were selected from multiple peptide fragments showing quantitative difference between in normal group and in patient group (see Table 2). PCA (principle component analysis) was performed with the selected 4 specific peptides. As a result, the difference between normal group and patient group was more clearly confirmed (see FIG. 3). The selected peptides were analyzed by ROC. As a result, it was confirmed that those peptides showed high sensitivity and specificity to distinguish patient group from normal group (see Table 3). Therefore, it was confirmed that by comparing quantitative difference of the selected 4 specific peptides, cancer patient group can be distinguished from normal group without comparing all the proteins quantitatively (see FIG. 4).

The present inventors performed sequence analysis with those selected 4 specific peptides. As a result, the sequence was confirmed to be related to N-linked protein glycosylation specific motif (N-X-S/T motif) (see Table 4). Difference of protein hydrolysis caused by protein glycosylation was confirmed by quantitative analysis with those selected specific peptides. Based on the result, cancer patient blood sample can be distinguished from normal blood sample (see FIG. 3). The selected specific peptides of the present invention thus can be effectively used as marker peptides available for diagnosis, prognosis, or verification of cancer using human blood sample.

The present invention also provides a diagnostic kit for cancer comprising the antibody specifically binding to one or more glycosylation related peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4.

The kit enables diagnosis and screening of cancer by confirming significant quantitative changes resulted from sugar chain changes caused by the treatment of hydrolase to the subject sample.

The cancer herein is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

The said 4 marker peptides or their isotope-labeled peptides can be additionally included in the kit as standard materials.

The antibody usable in the kit includes polyclonal antibody, monoclonal antibody, and epitope linkable fragment, etc. The polyclonal antibody can be produced by the conventional method comprising the steps of injecting one of the peptide markers to an animal; drawing blood from the animal; and obtaining serum containing the antibody. Such polyclonal antibody can be produced using a host animal selected from the group consisting of goat, rabbit, sheep, monkey, horse, pig, cow, dog, etc, and purified by the conventional method well-known to those in the art. The monoclonal antibody herein can be prepared by any technique that can provide antibody molecules through continuous culture of cell line, which is exemplified by hybridoma technique, human B-cell hybridoma technique, and EBV-hybridoma technique (Kohler G et al., Nature 256:495-497, 1975; Kozbor D et al., J Immunol Methods 81:31-42, 1985; Cote R J et al., Proc Natl Acad Sci 80:2026-2030, 1983; and Cole S P et al., Mol Cell Biol 62:109-120, 1984), but not always limited thereto. Also, the antibody fragment containing the specific binding site to one of those peptide markers can be prepared (Huse W D et al., Science 254: 1275-1281, 1989). The method to prepare such peptide specific antibody having specific sequence is well known to those in the art.

The said antibody is preferably specific to the peptide before and/or after glycosylation changes, but not always limited thereto.

The antibody used in the kit of the present invention can be fixed on solid substrate to make post-steps such as washing or isolation easy. The solid substrate herein is exemplified by synthetic resin, nitrocellulose, glass board, metal board, glass fiber, microsphere, and microbead. The synthetic resin herein can be polyester, polyvinyl chloride, polystyrene, polypropylene, PVDF, or nylon.

The sample obtained from a subject is contacted with the antibody binding specifically to one of the peptide markers fixed on the solid substrate. At this time, the sample can be diluted properly before the contact.

After contacting the sample obtained from a subject with the antibody binding specifically to one of the peptide markers fixed on the solid substrate, unbound proteins are removed by washing, followed by detection of specific peptides using MALDI MS.

The kit of the present invention can additionally include antibody for detection binding specifically to the said peptide marker. The antibody for detection herein can be the conjugate labeled with coloring enzyme, fluorescent material, radio-isotope, and colloid, etc, and preferably secondary antibody binding specifically to the said marker. The coloring enzyme herein can be peroxidase, alkaline phosphatase, or acid phosphatase (ex: horseradish peroxidase). The fluorescent material herein can be fluorescein carboxylic acid (FCA), fluorescein isothiocyanate (FITC), fluorescein thiourea (FTH), 7-acetoxycourmarin-3-yl, fluorescein-5-yl, fluorescein-6-yl, 2′,7′-dichlorofluorescein-5-yl, 2′,7′-dichlorofluorescein-6-yl, dihydrotetramethylrhosamine-4-yl, tetramethylrhodamine-5-yl, tetramethylrhodamine-6-yl, 4,4-difluoro-5,7-dimethyl-4-bora-3a,4a-deaza-s-indacene-3-ethyl or 4,4-difluoro-5,7-diphenyl-4-bora-3a,4a-deaza-s-indacene-3-ethyl.

The kit of the present invention can additionally include substrate to be reacted with coloring enzyme, and washing fluid or eluent to eliminate unbound proteins and to keep conjugated peptide markers only.

The present invention also provides a biochip for cancer diagnosis on which the antibody specifically binding to one or more glycosylation related peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 is integrated on the solid substrate.

The biochip enables diagnosis and screening of cancer by confirming significant quantitative changes resulted from sugar chain changes caused by the treatment of hydrolase to the subject sample.

The cancer herein is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

The biomolecule herein is preferably antibody or aptamer, but not always limited thereto. The said biomolecule indicates not only small molecule such as primary metabolite, secondary metabolite and natural substance but also organic molecule produced by living organism such as protein, polysaccharide and nucleic acid. The aptamer herein indicates oligonucleotide or peptide binding to the specific target molecule.

The solid substrate herein is preferably selected from the group consisting of plastic, glass, metal, and silicon, but not always limited thereto.

The present invention also provides a use of the antibody specifically binding to any peptide selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 for the preparation of the diagnostic kit for cancer.

The cancer herein is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

In addition, the present invention provides a use of a biomolecule, which can be obtained from the blood sample of a subject, specifically binding to the combination of one or more peptides selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4 for the preparation of the biochip for cancer diagnosis.

The cancer herein is preferably the one selected from the group consisting of colon cancer, stomach cancer, lung cancer, liver cancer, uterine cancer, breast cancer, prostatic cancer, thyroid cancer, and pancreatic cancer, and more preferably liver cancer, but not always limited thereto.

The biomolecule herein is preferably antibody or aptamer, but not always limited thereto. The said biomolecule indicates not only small molecule such as primary metabolite, secondary metabolite and natural substance but also organic molecule produced by living organism such as protein, polysaccharide and nucleic acid. The aptamer herein indicates oligonucleotide or peptide binding to the specific target molecule.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Sample Preparation

The present inventors obtained proteome from normal blood and liver cancer patient blood (Yonsei University Severance Hospital, Korea) as shown in Table 1, followed by pretreatment process as generally known for the convenience of purification.

TABLE 1

| Patient | | | | | | |
|---|---|---|---|---|---|---|
| No. | Age | Gender | Stage | Necrosis | Cause | Pathology |
| 1 | 25 | Male | 3 | 0 | HBV | Hepatitis |
| 2 | 61 | Male | 1~2 | 0 | HCV | Chronic |
| | | | | | | hepatitis |
| 3 | 72 | Male | 2 | 10 | HBV | Chronic |
| | | | | | | hepatitis |
| 4 | 46 | Female | 3 | 0 | HBV | Cirrhosis |
| 5 | 66 | Female | 1 | 0 | HCV | Cirrhosis |
| 6 | 46 | Male | 1~2 | 30 | HBV | Cirrhosis |
| 7 | 59 | Male | 2 | 30 | HBV | Cirrhosis |

Plasma proteome is composed of at least 50,000 constituents and the density of protein components is very dynamic (1˜10¹²). So, a biomarker candidate protein, especially existing at a very low concentration and taking less than 10%, is hard to detect and analyze quantitatively by liquid chromatography-mass spectrometry (LC/MS/MS) having the detection limit of 10⁴˜10⁶. Therefore, to minimize complexity of the sample for efficient detection of a disease biomarker in serum, protein removal column (ex, MARS, Multiple Affinity Removal System) was first used to eliminate those proteins taking about 90% or more of plasma proteome, for example albumin, IgG, IgA, transferrin, and haptoglobin, and then the resultant proteome was used. The obtained plasma proteome was purified by acetone precipitation or molecular weight cut-off (MWCO). The purified protein was dissolved in Tris-HCl buffer (pH=8.00), followed by quantification by Bradford Assay. Then, equal amount of total protein was prepared from each of normal group and patient group. Dithiothreitol (DTT, mM) was added to the prepared protein sample, followed by reaction at 60 for 30 minutes. As a result, disulfide bond at cysteine site was reduced, leading to protein denaturation. The reduced cysteine site was blocked by reacting with IAA (iodoacetamide) alkylation reagent at room temperature for 30 minutes in a dark room. The cysteine site protected protein was digested by reacting with trypsin at 37 for 10 hours. The peptides obtained from hydrolysis of protein were dried by using vacuum dryer. The dried peptides of each normal and patient group were dissolved in the same volume of buffer to prepare the peptide samples at the same concentration. As an internal standard material, yeast originated glucose-6-phosphate dehydrogenase (GPD) peptide was added to all the samples at the same concentration, followed by the experiment illustrated in <Example 2>.

### Example 2

### Peptide Analysis

To isolate and purify the samples prepared in <Example 1>, trap column (C18, 5 μm, 180 μm×20 mm, Waters) and analyzing column (BEH, C18, 1.7 μm, 75 μm×15 cm, Waters) were connected to nano-HPLC (Waters) and used. ESI-MS/MS was performed with the isolated/purified samples using electrospray ionization (ESI) mass spectrometer [Premier (quadruple-time of flight (Q-TOF)), Waters, UK] connected directly to nano-HPLC. Mixed peptides were obtained by hydrolysis using trypsin from each sample proteome, which were loaded on liquid chromatography connected with mass spectrometer (LC-ESI/MS/MS) at the same concentration of 5 μl. The sample proceeded to trap column (C18, 5 μm, 180 μm×20 mm) to eliminate salts therein. Then, complicated peptides were isolated by analyzing column (BEH, C18, 1.7 μm, 75 μm×15 cm). Samples of each time zone were detected as m/z value through mass spectrometer. Upon completion of the analysis, proteins were qualified by search engines such as Protein Expression System, MASCOT, and SEQUEST, etc. Those qualified peptides were confirmed by selected ion chromatogram based on the isolation time and m/z value obtained from the above mass spectrometry. To analyze quantitatively the qualified results of ESI-MS/MS by the label-free quantitative analysis method, it is important to know exact molecular weight of peptide and reproducibility of peptide isolation time. So, lock spray was loaded in Premier mass spectrometer to prevent electric sprayed ions isolated in liquid chromatography from being entered in the mass spectrometer and in the meantime to spray standard material (GFP, Glu-Fibrinopeptide B) whose molecular weight had been accurately identified only. As a result, the system was constructed to provide highly reliable results with reproducibility by correcting the molecular weight of each peptide more accurately. To perform quantitative analysis with normal group sample and patient group sample based on more reliable results with reproducibility, ESI-MS/MS was repeated three times.

### Example 3

### Qualitative and Quantitative Analysis

The results obtained in <Example 2> were qualified by the search engine MASCOT. Focused database was constructed by collecting all the qualified protein lists obtained from normal group and patient group. Qualitative/quantitative analysis was performed based on the focused database using Protein Expression System) (Waters, UK, version 2.1). The results of qualitative/quantitative analysis were exported to Excel, followed by principle component analysis (PCA). As a result, as shown in FIG. 2, patient group was clearly distinguished from normal group (FIG. 2). Therefore, it was confirmed that the peptide analysis method of the present invention is useful for the comparative screening of patient group from normal group.

Peptide pattern analysis was also performed with those proteins obtained from normal group and patient group based on the statistically treated results of PCA. As a result, specific peptides particularly showing quantitative changes and closely related to glycosylation site were selected among peptides belonging to the same protein, as shown in Table 2. All the selected peptides were confirmed to be related to N-linked glycosylation site in each glycoprotein (Table 4).

TABLE 2

| | | | | Standard |
|---|---|---|---|---|
| | | | | Deviation |
| | | | Normal | of Normal |
| | | | Group/ | Group/ |
| | Peptide | Normal | Patient | Patient |
| Protein | Sequence | Group | Group | Group |
| Afamin | TINPAVDHCCK | 1.00 | 3.67 | 3.67 ± 2.29 |
| precursor | (SEQ. ID. | | | |
| | NO: 1) | | | |
| Alpha 1 acid | TEDTIFLR | 1.00 | 1.64 | 1.64 ± 0.41 |
| glycoprotein | (SEQ. ID. | | | |
| 1 precursor | NO: 2) | | | |
| Isoform HMW | ENFLFLTPDCK | 1.00 | 1.64 | 1.64 ± 0.29 |
| of Kininogen | (SEQ. ID. | | | |
| 1 precursor | NO: 3) | | | |
| Vitronectin | GQYCYELDEK | 1.00 | 1.39 | 1.39 ± 0.24 |
| precursor | (SEQ. ID. | | | |
| | NO: 4) | | | |

Principle component analysis was also performed with the selected glycoprotein specific peptides alone by the same manner as described in the above. As a result, as shown in FIG. 3, patient group was more clearly distinguished from normal group, compared with the results shown in FIG. 2.

Sensitivity and specificity of each of the selected glycoprotein related specific peptide were presented as ROC curve (receiver operating characteristic curve), which are useful for distinguishment of patient group from normal group. As a result, as shown in Table 3 and FIG. 4, the selected specific peptides had high sensitivity and specificity (Table 3 and FIG. 4). Area in ROC curve indicates accuracy, and the area under the ROC curve (AUC) could be a tool to determine whether patient group could be distinguishable from normal group. As shown in Table 3, two peptides originated from afamin precursor and isoform HMW of kininogen 1 precursor provided excellent accuracy of at least 0.90. The peptide originated from alpha 1 acid glycoprotein 1 precursor provided good accuracy of at least 0.80. Another peptide originated from vitronectin precursor provided fair accuracy of about 0.70. Therefore, those peptides could be used separately or together to distinguish patient group from normal group.

In conclusion, when normal group and patient group are comparatively screened, it is not necessary to compare all the peptides detected by ESI-MS/MS. Instead, for example in the case of liver cancer patients, it is possible to distinguish patient group from normal group by screening glycoprotein related specific peptides only.

TABLE 3

| | | AUC (Area | |
|---|---|---|---|
| | Peptide | under the | |
| Protein | Sequence | ROC curve) | MH+ |
| Afamin | TINPAVDHCCK | 0.952 | 1314.60 |
| precursor | (SEQ. ID. NO: | | |
| | 1) | | |
| Alpha 1 acid | TEDTIFLR | 0.833 | 994.52 |
| glycoprotein 1 | (SEQ. ID. NO: | | |
| precursor | 2) | | |
| Isoform HMW | ENFLFLTPDCK | 0.984 | 1383.66 |
| of Kininogen 1 | (SEQ. ID. NO: | | |
| precursor | 3) | | |
| Vitronectin | GQYCYELDEK | 0.762 | 1304.55 |
| | (SEQ. ID. NO: | | |
| precursor | 4) | | |

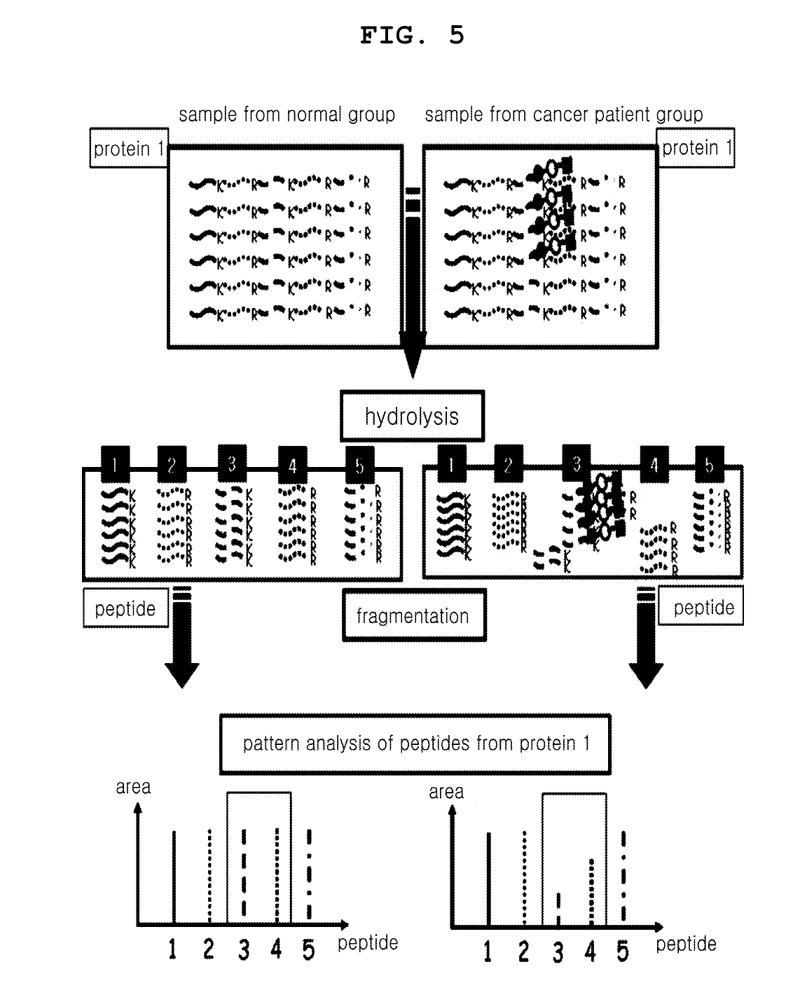

As shown in Table 4, the result of ESI-MS/MS suggests that the selected peptides are related to N-linked glycosylation in each protein (Table 4). When protein is glycosylated, steric hindrance effect occurs owing to the huge space taken by sugar chains. Thus, as shown in FIG. 5, the efficiency of hydrolysis of neighboring specific peptides is also affected. As a result, quantitative changes of the produced specific peptides are observed according to the changes of glycosylation and glycosylated structure in the region (FIG. 5).

TABLE 4

| | | | N-linked |
|---|---|---|---|
| | | | glycosylation |
| | | Peptide | related peptide |
| No. | Protein | Sequence | sequence |
| 1 | Afamin | TINPAVDHCCK | NRTINPAVDHCCK |
| | precursor | (SEQ. ID. NO: | |
| | | 1) | |
| 2 | Alpha 1 acid | TEDTIFLR | NKTEDTIFLR |
| | glycoprotein | (SEQ. ID. NO: | |
| | 1 precursor | 2) | |
| 3 | Isoform HMW | ENFLFLTPDCK | NCSKENFLFLTPDCK |
| | of Kininogen | (SEQ. ID. NO: | |
| | 1 precursor | 3) | |
| 4 | Vitronectin | GQYCYELDEK | NGSLFAFRGQYCYELD |
| | precursor | (SEQ. ID. NO: | EK |
| | | 4) | |

### INDUSTRIAL APPLICABILITY

As explained hereinbefore, the present invention provides a selection method of glycosylation specific peptides and a cancer diagnosis method using the selected peptides as markers, by which various cancers can be diagnosed using blood samples.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A screening method for cancer diagnosis marker, which comprises the following steps:
1) isolating total protein from the sample obtained from cancer patient;
2) purifying the isolated total protein by using mass protein removal column;
3) preparing hydrolyzed peptide fragment mixture by treating the purified protein with hydrolase;
4) quantitative-analyzing the hydrolyzed peptide fragment mixture;
5) screening those peptides showing significant change in the amount compared with that of a control; and
6) confirming if the selected peptides showing significant change in the amount are originated from glycoprotein.

2. The screening method for cancer diagnosis marker according to claim 1, wherein the cancer is characteristically selected from the group consisting of liver cancer, stomach cancer, colon cancer, lung cancer, uterine cancer, breast cancer, prostatic cancer, thyroid cancer and pancreatic cancer.

3. The screening method for cancer diagnosis marker according to claim 1, wherein the sample of step 1) is the one characteristically selected from the group consisting of cell, blood, serum, plasma, saliva, urine, cerebrospinal fluid, follicular fluid, breast milk, lens fluid and pancreatic juice.

4. The screening method for cancer diagnosis marker according to claim 1, wherein the purified protein of step 3) is characteristically pretreated with dithiothreitol (DTT) and iodoacetamide (IAA) before hydrolysis.

5. The screening method for cancer diagnosis marker according to claim 1, wherein the hydrolase of step 3) is characteristically selected from the group consisting of Arg-C, Asp-N, Glu-C, Lys-C, chymotrypsin and trypsin.

6. The screening method for cancer diagnosis marker according to claim 1, wherein the mass spectrometry of step 4) is characteristically performed by liquid chromatography-mass spectrometry (LG-MS).

7. The screening method for cancer diagnosis marker according to claim 1, wherein the glycoprotein originated peptide of step 6) characteristically has glycosylation site within the range of 8 amino acids from hydrolysis site either at N-terminal or C-terminal of the amino acid sequence.

8. The screening method for cancer diagnosis marker according to claim 1, wherein the glycoprotein originated peptide of step 6) is characteristically selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4.

9. A method for providing information for cancer diagnosis, which comprises the following steps:
1) isolating total protein from the sample obtained from a subject;
2) purifying the isolated total protein by using mass protein removal column;
3) preparing hydrolyzed peptide fragment mixture by treating the purified protein with hydrolase;
4) quantitative-analyzing the hydrolyzed peptide fragment mixture;
5) screening those peptides showing significant change in the amount compared with that of a control;
6) confirming if the selected peptides showing significant change in the amount are originated from glycoprotein; and
7) Judging or diagnosing cancer or high risk of cancer to the subject when the peptides showing significant change in the amount are confirmed to be originated from glycoprotein.

10. The method according to claim 9, wherein the sample of step 1) is the one characteristically selected from the group consisting of cell, blood, serum, plasma, saliva, urine, cerebrospinal fluid, follicular fluid, breast milk, lens fluid and pancreatic juice.

11. The method according to claim 9, wherein the hydrolase of step 3) is characteristically selected from the group consisting of Arg-C, Asp-N, Glu-C, Lys-C, chymotrypsin and trypsin.

12. The method according to claim 9, wherein the glycoprotein originated peptide of step 7) characteristically has glycosylation site within the range of 8 amino acids from hydrolysis site either at N-terminal or C-terminal of the amino acid sequence.

13. The method according to claim 9, wherein the glycoprotein originated peptide of step 7) is characteristically selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4.

14. A diagnostic kit for cancer comprising the antibody specifically binding to the peptide selected from the group consisting of afamin precursor having the amino acid sequence represented by SEQ. ID. NO: 1, alpha 1 acid glycoprotein 1 having the amino acid sequence represented by SEQ. ID. NO: 2, isoform HMW of kininogen 1 precursor having the amino acid sequence represented by SEQ. ID. NO: 3, and vitronectin precursor having the amino acid sequence represented by SEQ. ID. NO: 4.

15. The diagnostic kit for cancer according to claim 14, wherein the cancer is characteristically selected from the group consisting of liver cancer, stomach cancer, colon cancer, lung cancer, uterine cancer, breast cancer, prostatic cancer, thyroid cancer and pancreatic cancer.

16.-22. (canceled)
